(19) United States
(12) Patent Application Publication
(10) Pub. No.: US 2023/0130263 A1
(43) Pub. Date: Apr. 27, 2023

(54) Method For Recognizing Abnormal Sleep Audio Clip, Electronic Device

(71) Applicant: BAIDU INTERNATIONAL TECHNOLOGY (SHENZHEN) CO., LTD., Shenzhen (CN)

(72) Inventors: Jiping Wu, Shenzhen (CN); Xiaolong Lin, Shenzhen (CN); Jainmin Wu, Shenzhen (CN); Peicong Chen, Shenzhen (CN); Shengqiang Ruan, Shenzhen (CN); Guangjin Song, Shenzhen (CN); Zhenpeng Zhan, Shenzhen (CN); Gang Qiao, Shenzhen (CN)

(21) Appl. No.: 18/088,005

(22) Filed: Dec. 23, 2022

(30) Foreign Application Priority Data
Dec. 24, 2021 (CN) 202111603381.9

(51) Int. Cl. A61B 5/00; G06F 3/16

(52) U.S. Cl. CPC A61B 5/4818; A61B 5/743; A61B 5/4812; G06F 3/16

(57) ABSTRACT

A method for recognizing an abnormal sleep audio clip, includes: obtaining a plurality of initial audio clips collected by a sensor, and determining a target audio clip matching a preset sleep state from the initial audio clips; determining first snore information before the target audio clip and second snore information after the target audio clip based on the initial audio clips; determining a confidence value for the target audio clip based on the first snore information and the second snore information; and determining whether the target audio clip is the abnormal sleep audio clip based on the confidence value of the target audio clip.

### CROSS-REFERENCE TO RELATED APPLICATION

This application claims priority to Chinese Patent Application No. 202111603381.9, filed on Dec. 24, 2021, the entire disclosure of which is incorporated herein by reference.

### TECHNICAL FIELD

The disclosure relates to deep learning techniques in the artificial intelligence technologies, in particular to a method for recognizing an abnormal sleep audio clip, and an electronic device.

### BACKGROUND

Sleep quality is very important to people and in order for a user to be fully aware of his sleep state, sleep monitoring of the user can be performed.

In a first solution, the sleep monitoring can be performed by a professional using a special device to obtain a Polysomnography (PSG). In a second solution, sleep monitoring can be performed by a smartphone having a general model arranged thereon, the data collected by the smartphone is input into the model, to obtain a sleep monitoring result of a user.

### SUMMARY

According to a first aspect of the disclosure, a method for recognizing an abnormal sleep audio clip is provided. The method includes:
obtaining a plurality of initial audio clips collected by a sensor, and determining a target audio clip matching a preset sleep state from the initial audio clips, in which the preset sleep state represent an abnormal sleep state;
determining first snore information before the target audio clip and second snore information after the target audio clip based on the initial audio clips;
determining a confidence value for the target audio clip based on the first snore information and the second snore information, in which the confidence value is used to represent a possibility that the target audio clip is an abnormal sleep audio clip; and
determining whether the target audio clip is the abnormal sleep audio clip based on the confidence value of the target audio clip.

According to a second aspect of the disclosure, an electronic device is provided. The electronic device includes: at least one processor and a memory communicatively coupled to the at least one processor. The memory stores instructions executable by the at least one processor, and when the instructions are executed by the at least one processor, the at least one processor is caused to implement the method according to the first aspect of the disclosure.

According to a third aspect of the disclosure, a non-transitory computer-readable storage medium having computer instructions stored thereon is provided. The computer instructions are configured to cause a computer to implement the method according to the first aspect of the disclosure.

It should be understood that the content described in this section is not intended to identify key or important features of the embodiments of the disclosure, nor is it intended to limit the scope of the disclosure. Additional features of the disclosure will be easily understood based on the following description.

### BRIEF DESCRIPTION OF THE DRAWINGS

The drawings are used to better understand the solution and do not constitute a limitation to the disclosure, in which:

FIG. 1 is a flowchart of a method for recognizing an abnormal sleep audio clip according to an embodiment of the disclosure.

FIG. 2 is a flowchart of a method for recognizing an abnormal sleep audio clip according to another embodiment of the disclosure.

FIG. 3 is a schematic diagram of a sleep curve according to an embodiment of the disclosure.

FIG. 4 is a block diagram of an apparatus for recognizing an abnormal sleep audio clip according to an embodiment of the disclosure.

FIG. 5 is a block diagram of an apparatus for recognizing an abnormal sleep audio clip according to another embodiment of the disclosure.

FIG. 6 is a block diagram of an electronic device used to implement the method for recognizing an abnormal sleep audio clip according to an embodiment of the disclosure.

### DETAILED DESCRIPTION

The following describes the embodiments of the disclosure with reference to the accompanying drawings, which includes various details of the embodiments of the disclosure to facilitate understanding, which shall be considered merely exemplary. Therefore, those of ordinary skill in the art should recognize that various changes and modifications can be made to the embodiments described herein without departing from the scope and spirit of the disclosure. For clarity and conciseness, descriptions of well-known functions and structures are omitted in the following description.

A smartphone can be used to collect data, such as audio data, and then the collected data can be input into a model provided in the smartphone, so that the model outputs a sleep state, thereby achieving the purpose of performing sleep monitoring of a user.

However, the sleep state determined based only on the output result of the model is not accurate enough, and abnormal sleep data cannot be accurately recognized. Therefore, in the solution provided by the disclosure, a target audio clip that may belong to an abnormal sleep state is identified among a plurality of collected audio clips, and it is determined whether the target audio clip is indeed an abnormal sleep audio clip based on snore information before and after the target audio clip, thereby enabling accurate monitoring the users’ sleep state and recognizing the abnormal sleep audio clip.

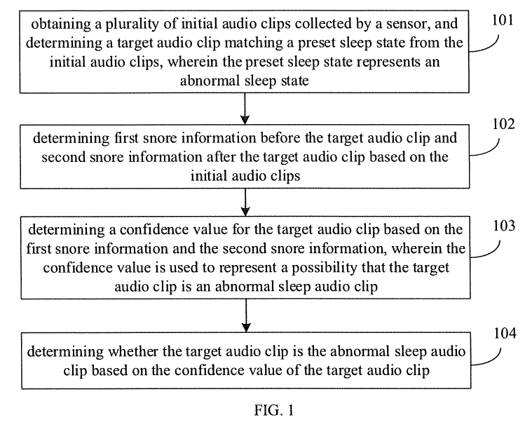

FIG. 1 is a flowchart of a method for recognizing an abnormal sleep audio clip according to an embodiment of the disclosure.

As illustrated in FIG. 1, the method for recognizing an abnormal sleep audio clip according to the disclosure further includes the following blocks.

At block 101, a plurality of initial audio clips collected by a sensor are obtained, and a target audio clip matching a preset sleep state is determined from the initial audio clips, in which the preset sleep state represents an abnormal sleep state.

The disclosure provides a solution that can be executed by an electronic device having a computing capability, which may be, for example, a smartphone.

There may be a plurality of sensors provided in the smartphone, for example, the sensors may include a microphone. The microphone may be used to collect audio clips, for example, an initial audio clip of 10 seconds may be collected.

The smartphone may implement the method provided in the disclosure while the user is sleeping. For example, other solutions can be used to determine whether the user is sleeping, and if the user is sleeping, the method of the disclosure may be implemented. Or, the method of the disclosure may be implemented when reaching a preset time point. For example, the method of the disclosure may be implemented at 10:00 p.m. In this way, various sleep states of the user can be determined and one or more abnormal sleep audio clip can be recognized.

In detail, when implementing the method of the disclosure, the smartphone may acquire the initial audio clips captured by a sensor, such as the initial audio clips captured by the microphone.

Further, the smartphone may obtain the data captured by the microphone at regular intervals, thereby obtaining the initial audio clips. For example, the data captured by the microphone can be acquired every 10 seconds, thus the initial audio clips of 10 seconds are obtained.

In practice, the smartphone can process each of the initial audio clips to determine a sleep state of each initial audio clip. For example, an initial audio clip is determined to be a hypopnea state, an apnea state, a continuous breathing state, and a continuous snoring state.

Hypopnea refers to a decrease in strength (amplitude) of the respiratory airflow during sleep by more than 50% compared with a basic level, accompanied by a decrease in blood oxygen saturation by more than or equal to 4% compared with the basic level or slight awakening. Apnea syndrome refers to a sudden interruption of loud snoring, where the user breathes forcefully but ineffectively, and cannot breathe at all, the user wakes up after a few seconds or even tens of seconds, gasps loudly, and the airway is forced open, and then the user continues to breathe.

If the user is in a continuous breathing state or a continuous snoring state while sleeping, the user is in a normal sleep state. If the user is in a hypopnea state or an apnea state during sleep, it indicates that there is an abnormal sleep state.

If an initial audio clip matches the preset abnormal sleep state, it is determined that the initial audio clip is the target audio clip.

In detail, a model for recognizing a sleep state may be preset, and each initial audio clip is input into the model to obtain a sleep state of each initial audio clip, thus the target audio clip can be determined.

At block 102, first snore information before the target audio clip and second snore information after the target audio clip are determined based on the initial audio clips.

Further, after determining the target audio clip, the target audio clip may be considered to be a suspected abnormal sleep audio clip, and it may be further determined whether the target audio clip is indeed an abnormal sleep audio clip.

In practice, the first snore information of the user before the target audio clip and the second snore information of the user after the target audio clip can be determined, so that it is determined whether the target audio clip is indeed an abnormal sleep clip based on the first snore information and the second snore information.

For example, a first initial audio clip before the target audio clip may be determined, and the first snore information may be determined based on the first initial audio clip. A second initial audio clip after the target audio clip may be determined, and the second snore information may be determined based on the second initial audio clip.

For example, the first snore information may be determined based on snore intensities at a plurality of moments in the first initial audio clip, and the second snore information may be determined based on snore intensities at a plurality of moments in the second initial audio clip. Alternatively, a snore intensity at the last moment in the first initial audio clip may be determined as the first snore information, and a snore intensity at a starting moment in the second initial audio clip may be determined as the second snore information.

At block 103, a confidence value for the target audio clip is determined based on the first snore information and the second snore information, in which the confidence value is used to represent a possibility that the target audio clip is an abnormal sleep audio clip.

In detail, the confidence value for the target audio clip may be determined based on the first snore information and the second snore information, and the confidence value is used to represent a possibility that the target audio clip belongs to an abnormal sleep audio clip.

For example, the greater a difference in intensity between the first snore information and the second snore information, the greater the determined confidence value is, and the greater the possibility that the target audio clip is the abnormal sleep audio clip is. Otherwise, the smaller the difference in intensity between the first snore information and the second snore information, the smaller the determined confidence value is, and the smaller the possibility that the target audio clip is the abnormal sleep audio clip is.

At block 104, it is determined whether the target audio clip is the abnormal sleep audio clip based on the confidence value of the target audio clip.

In detail, the smartphone may determine whether the target audio clip is the abnormal sleep audio clip based on the confidence value of the target audio clip.

For example, a confidence threshold may be set in advance, and if the determined confidence value of the target audio clip is greater than the confidence threshold, it may be determined that the target audio clip is an abnormal sleep audio clip, otherwise, it is determined that the target audio clip is not an abnormal sleep audio clip.

The method for recognizing an abnormal sleep audio clip provided in the present disclosure includes: obtaining the plurality of initial audio clips collected by the sensor, and determining the target audio clip matching the preset sleep state from the initial audio clips, in which the preset sleep states represent abnormal sleep states; for each of the target audio clips, determining the first snore information before the target audio clip and the second snore information after the target audio clip based on the initial audio clips; determining the confidence value for the target audio clip based on the first snore information and the second snore information, in which the confidence value is used to represent a possibility that the target audio clip is an abnormal sleep audio clip; and determining whether the target audio clip is the abnormal sleep audio clip based on the confidence value of the target audio clip. According to the method for recognizing an abnormal sleep audio clip of the disclosure, the target audio clip that may be an abnormal sleep audio clip can be initially recognized among the plurality of initial audio clips, and then it is determined whether the target audio clip is the abnormal sleep audio clip based on the snore information before and after the target audio clip, thus the abnormal sleep audio clip can be accurately recognized among the plurality of initial audio clips.

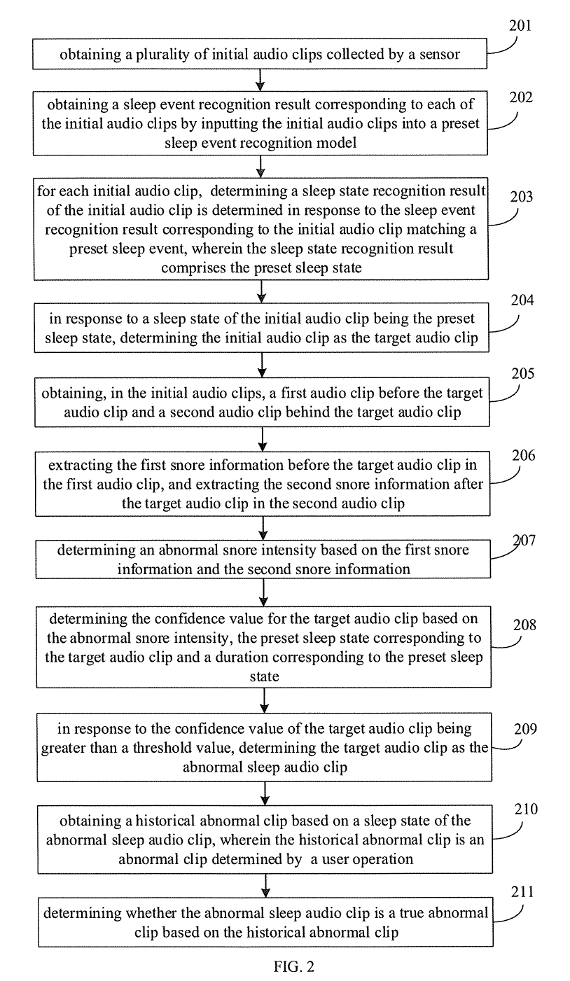

FIG. 2 is a flowchart of a method for recognizing an abnormal sleep audio clip according to another embodiment of the disclosure.

As illustrated in FIG. 2, the method for recognizing an abnormal sleep audio clip according to the disclosure includes the following blocks.

At block 201, a plurality of initial audio clips collected by a sensor are obtained.

The mode of obtaining the initial audio clips at block 201 is similar to the mode of obtaining the initial audio clips at block 101.

At block 202, a sleep event recognition result corresponding to each of the initial audio clips is obtained by inputting the initial audio clips into a preset sleep event recognition model.

The sleep event recognition model can be set up in a smartphone, and the sleep event recognition model may be pre-trained by machine learning techniques. For example, a plurality of audio data for training can be collected in advance, and sleep events can be labeled on the plurality of audio data correspondingly one by one, and the model can be trained using the audio data labeled with sleep events, so that the sleep event recognition model that can recognize a sleep event for audio data is obtained.

The initial audio clip may be input into the preset sleep event recognition model, which is capable of outputting a sleep event for the initial audio clip.

In detail, sleep events may include snoring events, sleep talking events, breathing events, and ambient sound events.

At block 203, for each initial audio clip, a sleep state recognition result of the initial audio clip is determined in response to the sleep event recognition result corresponding to the initial audio clip matching a preset sleep event, in which the sleep state recognition result includes the preset sleep state.

Further, if it is determined that the sleep event recognition result for the initial audio clip matches the preset sleep event, then it is possible that there is an abnormal sleep clip in the initial audio clip, therefore the sleep state of that initial audio clip may be further identified.

If it is determined that the sleep event recognition result for the initial audio clip does not match the preset sleep event, it indicates that there is no possibility of an abnormal sleep clip in the initial audio clip, therefore, no further detection of its sleep state is required.

Through this implementation, it is possible to recognize audio clips whose sleep states need to be identified from the initial audio clips, and only the sleep states of the initial audio clips that match the preset sleep event are to be identified, thereby reducing the amount of audio data whose sleep states are to be identified and increasing the speed of data processing.

If the sleep event recognition result for the initial audio clip matches the preset sleep event, the sleep state recognition result of the initial audio clip may be further determined.

An audio feature of the initial audio clip can be extracted. The sleep state recognition result of the initial audio clip can be determined based on the audio feature. The audio feature of the initial audio clip can be processed to obtain a more accurate sleep state recognition result of the initial audio clip.

In detail, the audio features, such as F-Bank, MFCC (Mel-frequency cepstral coefficient) and spectral energy, of the initial audio clip can be extracted and then a classification model obtained by pre-training can be used to classify the audio features to obtain a classification result. The classification result can include recognition results for a variety of sleep states. The classification model can be, for example, a random forest, a SVM (Support Vector Machine), a decision tree and other models.

The sleep state can include, for example, hypopnea, apnea, continuous breathing and continuous snoring. The classification model can process the audio features of the initial audio clip to obtain a probability that the initial audio clip belongs to each sleep state, and then the sleep state recognition result for the initial audio clip is obtained.

The preset sleep event includes a snoring event and a breathing event. The preset sleep state includes hypopnea and apnea.

Hypopnea and apnea sleep states are abnormal sleep states, and it is possible to detect the existence of hypopnea and apnea sleep states in sleep clips labeled with snoring events or breathing events.

Therefore, audio clips containing snoring events and breathing events are identified firstly in the respective initial audio clips, and then audio clips containing hypopnea and apnea sleep states are identified in these audio clips, thereby enabling accurate identification of abnormal sleep audio clips of hypopnea and apnea.

At block 204, in response to a sleep state of the initial audio clip being the preset sleep state, the initial audio clip is determined as the target audio clip.

In practice, if the sleep state of the initial audio clip is the preset sleep state, it may be determined that the initial audio clip may have an abnormal sleep state, thus the initial audio clip may be identified as the target audio clip.

At block 205, in the initial audio clips, a first audio clip before the target audio clip and a second audio clip behind the target audio clip are obtained.

The determined target audio clip is possible to be an abnormal sleep audio clip, and in order to more accurately determine whether it is an abnormal sleep audio clip, the first audio clip before the target audio clip and the second audio clip behind the target audio clip can be obtained.

In detail, each initial audio clip obtained by the smartphone may have corresponding time information. For example, clip 1, clip 2, and clip 3 are obtained, and the three clips have consecutive time information, if clip 2 is the target audio clip, clip 1 may be identified as the first audio clip before the target audio clip, and clip 3 may be identified as the second audio clip after the target audio clip.

Further, the snore information before and after the target audio clip may be determined based on other audio clips before and after the target audio clip, thereby more accurately determining whether the target audio clip is an abnormal sleep audio clip.

At block 206, the first snore information before the target audio clip is extracted in the first audio clip, and the second snore information after the target audio clip is extracted in the second audio clip.

In practical application, the smartphone may extract the first snore information in the first audio clip, for example, the snore information at each time point in the first audio clip may be processed to obtain the first snore information. For example, an average of the snore intensities at the respective time points may be taken as the first snore information.

The smartphone may extract the second snore information in the second audio clip, for example, by processing the snore information at each time point in the second audio clip to obtain the second snore information, or by determining an average of the snore intensities at the respective time points as the second snore information.

In detail, the smartphone may also use the snore intensity of the last moment in the first audio clip as the first snore information, and may use the snore intensity of the first moment in the second audio clip as the second snore information.

With this implementation, it is possible to determine the first snore information and the second snore information, thus it is determined whether the target audio clip is an abnormal sleep audio clip based on the first snore information and the second snore information. In this way, it is possible to determine whether the target audio clip is an abnormal sleep audio clip based on the target audio clip itself along with other audio clips before and after it, thereby making the recognition result more accurate.

At block 207, an abnormal snore intensity is determined based on the first snore information and the second snore information.

Further, the first snore information and the second snore information may be snore intensities, which may specifically be represented by a numerical value.

In practical application, an abnormal snore intensity before and after the target audio clip may be determined based on the first snore information and the second snore information, the abnormal snore intensity may specifically be a difference between the snore intensities before and after the target audio clip. For example, the difference between the intensity value in the first snore information and the intensity value in the second snore information may be taken as the abnormal snore intensity of the target audio clip.

At block 208, the confidence value for the target audio clip is determined based on the abnormal snore intensity, the preset sleep state corresponding to the target audio clip and a duration corresponding to the preset sleep state.

The smartphone may determine the confidence value for the target audio clip based on the abnormal snore intensity of the target audio clip, and its corresponding preset sleep state and duration.

For example, if the abnormal snore intensity of the target audio clip is p, the sleep state is hypopnea, and the duration of hypopnea is t, the confidence value for the target audio clip can be determined based on these three pieces of information. The values corresponding to different preset sleep states can be set in advance, and the method for determining the confidence values corresponding to different preset sleep states can also be set, so that the confidence value of the target audio clip can be determined.

The confidence value can take on a range of values from 0 to 1. The greater the confidence value, the greater the probability that the target audio clip is an abnormal sleep audio clip is.

Through this implementation, different preset sleep states, corresponding durations of the states and other factors are fully considered, and it is determined whether the target audio clip is an abnormal sleep audio clip based on these factors and the abnormal snore intensity of the target audio clip, thus the abnormal sleep audio clips can be accurately determined.

For example, if the duration of the preset sleep state in the target audio clip is relatively short, it is possible that the target audio clip is not an abnormal sleep audio clip. For another example, the results for two target audio clips with the same abnormal snore intensities and the same durations may be different if the preset sleep states corresponding to the two target audio clips are different. For example, one of the target audio clips may be identified as an abnormal sleep audio clip and another target audio clip is not identified as an abnormal sleep audio clip.

At block 209, in response to the confidence value of the target audio clip being greater than a threshold value, the target audio clip is determined as the abnormal sleep audio clip.

The threshold value may be preset, and it is determined whether the target audio clip is an abnormal sleep audio clip by comparing the confidence value of the target audio clip with the threshold value.

In detail, if the confidence value of the target audio clip is greater than the preset threshold value, it is determined that the target audio clip is an abnormal sleep audio clip.

After recognizing the target audio clip that is possible to be an abnormal sleep audio clip, it may be further determined whether the target audio clip is indeed an abnormal sleep audio clip based on the confidence value of the target audio clip, thereby recognizing the abnormal sleep audio clip more accurately.

At block 210, a historical abnormal clip is obtained based on a sleep state of the abnormal sleep audio clip, in which the historical abnormal clip is an abnormal clip determined by a user operation.

In detail, the smartphone may also acquire the historical abnormal clip.

While using the smartphone, the user can identify which of the initial audio clips collected by the smartphone are abnormal clips, specifically which are abnormal clips of hypopnea and which are abnormal clips of apnea.

Further, the smartphone may obtain the sleep state of the abnormal sleep audio clip and obtain the historical abnormal clip consistent with that sleep state. For example, if the currently identified sleep state of the abnormal sleep audio clip is a clip of hypopnea, one or more historical abnormal clips belonging to hypopnea may be obtained.

At block 211, it is determined whether the abnormal sleep audio clip is a true abnormal clip based on the historical abnormal clip.

In practice, it is further determined whether the abnormal sleep audio clip is a true abnormal clip based on the historical abnormal clip.

The historical abnormal clip is an abnormal sleep clip that is manually confirmed by the user and can therefore be assumed to be accurate. This historical abnormal clip can be used as standard data to reconfirm whether the historical abnormal clip identified by the smartphone is a true abnormal clip.

In detail, the smartphone may compare the historical abnormal clip with the abnormal sleep audio clip, and if the two are similar, it may determine that the abnormal sleep audio clip is a true abnormal clip, otherwise, it is determined that the abnormal sleep audio clip is not a true abnormal clip.

In this way, it is possible to more accurately identify the true abnormal clip based on the user’s historical abnormal clips. Moreover, the abnormal sleep clips are not exactly the same for different users, so the solution of the disclosure also enables personalized recognition based on data of different users, thereby further improving the recognition accuracy of the abnormal sleep audio clip.

Further, the smartphone may obtain a first audio feature of the historical abnormal clip, and a second audio feature of the abnormal sleep audio clip, and then may determine a similarity between the first audio feature and the second audio feature.

If the similarity between the first audio feature and the second audio feature satisfies a preset condition, the abnormal sleep audio clip is determined to be a true abnormal clip. By comparing the feature of the historical abnormal clip and the feature of the abnormal sleep audio clip and determining whether the two are similar, it is possible to determine whether the abnormal sleep audio clip is similar to the historical abnormal clip of the user, and then determine whether the abnormal sleep audio clip matches the abnormal sleep state of the user, for the purpose of personalized identification.

In an optional implementation, sleep aid device information and/or medical aid resource information corresponding to the abnormal sleep audio clip can be obtained and displayed based on the abnormal sleep audio clip.

In practice, the smartphone may also obtain information about the sleep aid device used to resolve the abnormal sleep state based on the abnormal sleep audio clip, and may also obtain information about the medical aid resource used to resolve the abnormal sleep state, and then display the obtained information, which causes the user to purchase the appropriate device by operating the smartphone, or learn about the appropriate medical aid resources.

Medical aid resources can, for example, be resources such as online consultation. A corresponding function portal can be displayed in the smartphone for accessing this function.

With the solution of the embodiments of the disclosure, it enables automatic recognition of sleep problems and provides solutions to sleep problems, thereby providing an integrated solution for sleep problems to the user.

In an optional implementation, a sleep curve is generated based on the abnormal sleep audio clips and the sleep curve is displayed, and the abnormal sleep audio clips are labeled in the sleep curve. The sleep curve is used to represent the sleep state of the user at various moments.

The solution provided in embodiments of the disclosure may also generate and display a sleep curve in which abnormal sleep audio clips may be labeled. For example, a sleep clip between a first time point and a second time point may be marked as an abnormal sleep audio clip for reference by the user.

In this way, the user can learn about their sleep state more intuitively, and the user can also operate on the sleep curve to play the abnormal sleep audio clips in the curve, and confirm or deny the abnormal sleep audio clips, so that the historical abnormal clips can be updated according to this operation, and the user’s sleep state can be more accurately and personally identified.

In detail, data can also be collected through sensors such as a gyroscope and a microphone of the smartphone. This solution implements sleep cycle monitoring for the user, mainly monitoring three sleep cycles: waking, light sleep and deep sleep, based on the artificial intelligence technology and normal human sleep cycle patterns.

The data collected by gyroscopes, microphones and smart wearable devices can be input into a preset deep learning model, through which sleep cycle estimation can be performed on the feature data. The user’s sleep cycle is predicted according to the estimated sleep cycle along with the pattern of the human sleep cycle (light sleep alternating with deep sleep, 90 to 100 minutes per cycle).

The sleep curve can also be generated based on this sleep cycle, so that the various sleep stages can also be labeled in the sleep curve, thus the sleep curve can be more informative, thereby enabling the user to visualize their sleep state.

In an optional embodiment, a sleep report may also be output daily or weekly, in which the user’s sleep state during this period is recorded.

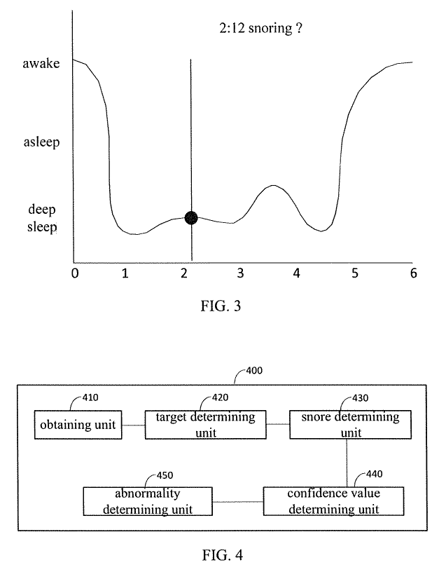

FIG. 3 is a schematic diagram of a sleep curve according to an embodiment of the disclosure.

As illustrated in FIG. 3, the smartphone can display a sleep curve as shown in FIG. 3. The user can also click on any position in the curve and a sleep state corresponding to the position can be displayed.

FIG. 4 is a block diagram of an apparatus for recognizing an abnormal sleep audio clip according to an embodiment of the disclosure.

As illustrated in FIG. 4, an apparatus 400 for recognizing an abnormal sleep audio clip of the disclosure includes: an obtaining unit 410, a target determining unit 420, a snore determining unit 430, a confidence value determining unit 440 and an abnormality determining unit 450.

The obtaining unit 410 is configured to obtain a plurality of initial audio clips collected by a sensor.

The target determining unit 420 is configured to determine a target audio clip matching a preset sleep state from the initial audio clips, in which the preset sleep state represent an abnormal sleep state.

The snore determining unit 430 is configured to determine first snore information before the target audio clip and second snore information after the target audio clip based on the initial audio clips.

The confidence value determining unit 440 is configured to determine a confidence value for the target audio clip based on the first snore information and the second snore information, in which the confidence value is used to represent a possibility that the target audio clip is an abnormal sleep audio clip.

The abnormality determining unit 450 is configured to determine whether the target audio clip is the abnormal sleep audio clip based on the confidence value of the target audio clip.

With the apparatus for recognizing an abnormal sleep audio clip according to the present disclosure, the target audio clip that is possible to be the abnormal sleep audio clip is initially recognized from the initial audio clips, and then the snore information before and after the target audio clip is used to determine whether the target audio clip is indeed an abnormal sleep audio clip, thereby accurately recognizing the abnormal sleep audio clips in the plurality of initial audio clip.

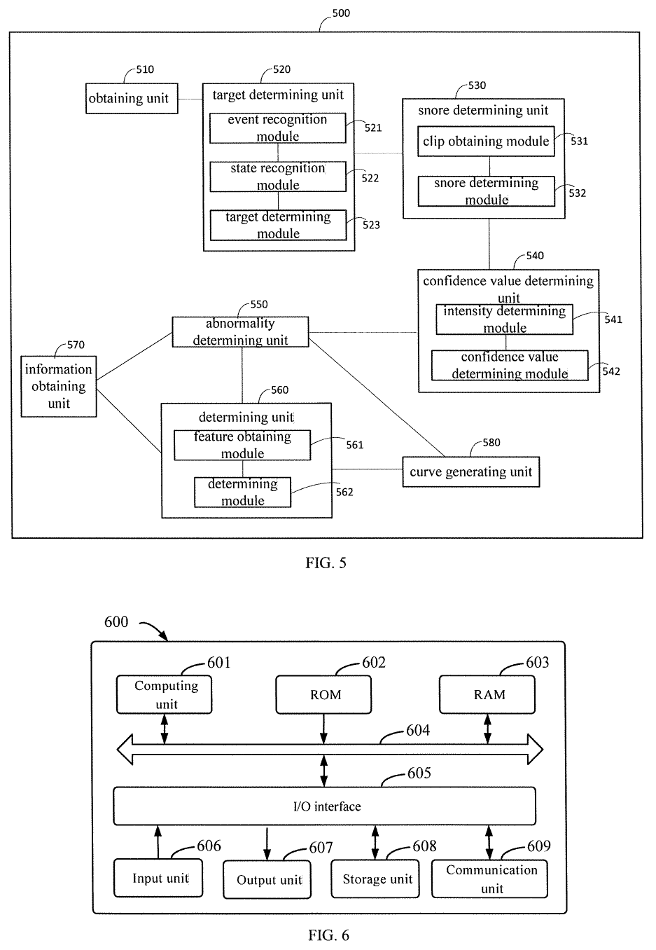

FIG. 5 is a block diagram of an apparatus 500 for recognizing an abnormal sleep audio clip according to another embodiment of the disclosure.

As illustrated in FIG. 5, in the apparatus 500 for recognizing an abnormal sleep audio clip of the disclosure, the obtaining unit 510 is similar to the obtaining unit 410 shown in FIG. 4, the target determining unit 520 is similar to the target determining unit 420 shown in FIG. 4, the snore determining unit 530 is similar to the snore determining unit 430 shown in FIG. 4, the confidence value determining unit 540 is similar to the confidence value determining unit 440 shown in FIG. 4, and the abnormality determining unit 550 is similar to the abnormality determining unit 450 shown in FIG. 4.

Optionally, the target determining unit 520 includes: an event recognition module 521, a state recognition module 522 and a target determining module 523.

The event recognition module 521 is configured to obtain a sleep event recognition result corresponding to each initial audio clip by inputting the initial audio clips into a preset sleep event recognition model.

The state recognition module 522 is configured to, for each initial audio clip, determining a sleep state recognition result of the initial audio clip in response to the sleep event recognition result corresponding to the initial audio clip matching a preset sleep event, in which the sleep state recognition result includes the preset sleep states.

The target determining module 523 is configured to determine the initial audio clip as the target audio clip in response to a sleep state of the initial audio clip being the preset sleep state.

Optionally, the state recognition module 522 is further configured to:
extract an audio feature of the initial audio clip; and
determine the sleep state identification result of the initial audio clip based on the audio feature of the initial audio clip.

Optionally, the preset sleep event includes a snoring event and a breathing event; and

The preset sleep state includes hypopnea and apnea.

Optionally, the snore determining unit 530 includes: a clip obtaining module 531 and a snore determining module 532.

The clip obtaining module 531 is configured to obtain, in the initial audio clips, a first audio clip before the target audio clip and a second audio clip after the target audio clip.

The snore determining module 532 is configured to extract, in the first audio clip, the first snore information before the target audio clip, and extract, in the second audio clip, the second snore information after the target audio clip.

Optionally, the confidence value determining unit 540 includes: an intensity determining module 541 and a confidence value determining module 542.

The intensity determining module 541 is configured to determine an abnormal snore intensity based on the first snore information and the second snore information.

The confidence value determining module 542 is configured to determine the confidence value for the target audio clip based on the abnormal snore intensity, the preset sleep state corresponding to the target audio clip and a duration corresponding to the preset sleep state.

Optionally, the abnormality determining unit 550 is configured to:

Determine that the target audio clip is the abnormal sleep audio clip in response to the confidence value of the target audio clip being greater than a threshold.

Optionally, in response to determining the target audio clip being the abnormal sleep audio clip, the apparatus further includes a determining unit 560, and the determining unit 560 is configured to:
obtain a historical abnormal clip based on a sleep state of the abnormal sleep audio clip, in which the historical abnormal clip is an abnormal clip determined by a user operation; and
determine whether the abnormal sleep audio clip is a true abnormal clip based on the historical abnormal clip.

Optionally, the determining unit 560 includes: a feature obtaining module 561 and a determining module 562.

The feature obtaining module 561 is configured to obtain a first audio feature of the historical abnormal clip and obtain a second audio feature of the abnormal sleep audio clip.

The determining module 562 is configured to determine that the abnormal sleep audio clip is the true abnormal clip in response to a similarity between the first audio feature and the second audio feature satisfying a preset condition.

Optionally, the apparatus further includes an information obtaining unit 570, and the information obtaining unit 570 is configured to:

Obtain and display sleep aid device information and/or medical aid resource information corresponding to the abnormal sleep audio clip.

Optionally, the apparatus further includes a curve generating unit 580, and the curve generating unit 580 is configured to:

generate a sleep curve based on the abnormal sleep audio clip and display the sleep curve, and label the abnormal sleep audio clip in the sleep curve, in which the sleep curve is used to represent sleep states of a user at different time points.

The disclosure provides a method for recognizing an abnormal sleep audio clip, an electronic device and a program product, and relates to deep learning techniques in artificial intelligence techniques, for accurately recognizing the abnormal sleep audio clips of the user.

The collection, storage, use, processing, transmission, provision and disclosure of the personal information of users involved in the technical solutions of the disclosure are handled in accordance with relevant laws and regulations and are not contrary to public order and morality.

According to the embodiments of the disclosure, the disclosure provides an electronic device, and a readable storage medium and a computer program product.

According to embodiments of the disclosure, the disclosure also provides a computer program product. The computer program product includes computer programs, and the computer programs are stored in a readable storage medium. At least one processor of the electronic device can read the computer programs from the readable storage medium, and the at least one processor is able to execute the computer programs to cause the electronic device to implement the solution provided by any of the above embodiments.

The disclosure provides a method for recognizing an abnormal sleep audio clip, an electronic device and a program product. The method includes: obtaining a plurality of initial audio clips collected by a sensor, and determining a target audio clip matching a preset sleep state from the initial audio clips, in which the preset sleep state represent an abnormal sleep state; determining first snore information before the target audio clip and second snore information after the target audio clip based on the initial audio clips; determining a confidence value for the target audio clip based on the first snore information and the second snore information, in which the confidence value is used to represent a possibility that the target audio clip is an abnormal sleep audio clip; and determining whether the target audio clip is the abnormal sleep audio clip based on the confidence value of the target audio clip. According to the solution of the disclosure, the target audio clip that is possible to be the abnormal sleep audio clip can be initially identified among the plurality of initial audio clips, and then it is determined whether the target audio clip is the abnormal sleep audio clip based on the snore information before and after the target audio clip, thus the abnormal sleep audio clips can be accurately recognized among the plurality of initial audio clips.

FIG. 6 is a block diagram of an example electronic device 600 used to implement the embodiments of the disclosure. Electronic devices are intended to represent various forms of digital computers, such as laptop computers, desktop computers, workbenches, personal digital assistants, servers, blade servers, mainframe computers, and other suitable computers. Electronic devices may also represent various forms of mobile devices, such as personal digital processing, cellular phones, smart phones, wearable devices, and other similar computing devices. The components shown here, their connections and relations, and their functions are merely examples, and are not intended to limit the implementation of the disclosure described and/or required herein.

As illustrated in FIG. 6, the electronic device 600 includes: a computing unit 601 performing various appropriate actions and processes based on computer programs stored in a read-only memory (ROM) 602 or computer programs loaded from the storage unit 608 to a random access memory (RAM) 603. In the RAM 603, various programs and data required for the operation of the device 600 are stored. The computing unit 601, the ROM 602, and the RAM 603 are connected to each other through a bus 604. An input/output (I/O) interface 605 is also connected to the bus 604.

Components in the device 600 are connected to the I/O interface 605, including: an inputting unit 606, such as a keyboard, a mouse; an outputting unit 607, such as various types of displays, speakers; a storage unit 608, such as a disk, an optical disk; and a communication unit 609, such as network cards, modems, and wireless communication transceivers. The communication unit 609 allows the device 600 to exchange information/data with other devices through a computer network such as the Internet and/or various telecommunication networks.

The computing unit 601 may be various general-purpose and/or dedicated processing components with processing and computing capabilities. Some examples of computing unit 601 include, but are not limited to, a CPU, a graphics processing unit (GPU), various dedicated AI computing chips, various computing units that run machine learning model algorithms, and a digital signal processor (DSP), and any appropriate processor, controller and microcontroller. The computing unit 601 executes the various methods and processes described above, such as the method for recognizing an abnormal sleep audio clip. For example, in some embodiments, the above method may be implemented as a computer software program, which is tangibly contained in a machine-readable medium, such as the storage unit 608. In some embodiments, part or all of the computer program may be loaded and/or installed on the device 600 via the ROM 602 and/or the communication unit 609. When the computer program is loaded on the RAM 603 and executed by the computing unit 601, one or more steps of the method described above may be executed. Alternatively, in other embodiments, the computing unit 601 may be configured to perform the method in any other suitable manner (for example, by means of firmware).

Various implementations of the systems and techniques described above may be implemented by a digital electronic circuit system, an integrated circuit system, Field Programmable Gate Arrays (FPGAs), Application Specific Integrated Circuits (ASICs), Application Specific Standard Products (ASSPs), System on Chip (SOCs), Load programmable logic devices (CPLDs), computer hardware, firmware, software, and/or a combination thereof. These various embodiments may be implemented in one or more computer programs, the one or more computer programs may be executed and/or interpreted on a programmable system including at least one programmable processor, which may be a dedicated or general programmable processor for receiving data and instructions from the storage system, at least one input device and at least one output device, and transmitting the data and instructions to the storage system, the at least one input device and the at least one output device.

The program code configured to implement the method of the disclosure may be written in any combination of one or more programming languages. These program codes may be provided to the processors or controllers of general-purpose computers, dedicated computers, or other programmable data processing devices, so that the program codes, when executed by the processors or controllers, enable the functions/operations specified in the flowchart and/or block diagram to be implemented. The program code may be executed entirely on the machine, partly executed on the machine, partly executed on the machine and partly executed on the remote machine as an independent software package, or entirely executed on the remote machine or server.

In the context of the disclosure, a machine-readable medium may be a tangible medium that may contain or store a program for use by or in combination with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of machine-readable storage medium include electrical connections based on one or more wires, portable computer disks, hard disks, random access memories (RAM), read-only memories (ROM), electrically programmable read-only-memory (EPROM), flash memory, fiber optics, compact disc read-only memories (CD-ROM), optical storage devices, magnetic storage devices, or any suitable combination of the foregoing.

In order to provide interaction with a user, the systems and techniques described herein may be implemented on a computer having a display device (e.g., a Cathode Ray Tube (CRT) or a Liquid Crystal Display (LCD) monitor for displaying information to a user); and a keyboard and pointing device (such as a mouse or trackball) through which the user can provide input to the computer. Other kinds of devices may also be used to provide interaction with the user. For example, the feedback provided to the user may be any form of sensory feedback (e.g., visual feedback, auditory feedback, or haptic feedback), and the input from the user may be received in any form (including acoustic input, voice input, or tactile input).

The systems and technologies described herein can be implemented in a computing system that includes background components (for example, a data server), or a computing system that includes middleware components (for example, an application server), or a computing system that includes front-end components (for example, a user computer with a graphical user interface or a web browser, through which the user can interact with the implementation of the systems and technologies described herein), or include such background components, intermediate computing components, or any combination of front-end components. The components of the system may be interconnected by any form or medium of digital data communication (e.g., a communication network). Examples of communication networks include: local area network (LAN), wide area network (WAN), and the Internet.

The computer system may include a client and a server. The client and server are generally remote from each other and interacting through a communication network. The client-server relation is generated by computer programs running on the respective computers and having a client-server relation with each other. The server may be a cloud server, also known as a cloud computing server or a cloud host, which is a host product in the cloud computing service system, to solve defects such as difficult management and weak business scalability in the traditional physical host and Virtual Private Server (VPS) service. The server may also be a server of a distributed system, or a server combined with a block-chain.

It should be understood that the various forms of processes shown above can be used to reorder, add or delete steps. For example, the steps described in the disclosure could be performed in parallel, sequentially, or in a different order, as long as the desired result of the technical solution disclosed in the disclosure is achieved, which is not limited herein.

The above specific embodiments do not constitute a limitation on the protection scope of the disclosure. Those skilled in the art should understand that various modifications, combinations, sub-combinations and substitutions can be made according to design requirements and other factors. Any modification, equivalent replacement and improvement made within the spirit and principle of this application shall be included in the protection scope of this application.

What is claimed is:

1. A method for recognizing an abnormal sleep audio clip, comprising:
obtaining a plurality of initial audio clips collected by a sensor, and determining a target audio clip matching a preset sleep state from the initial audio clips, wherein the preset sleep state represent an abnormal sleep state;
determining first snore information before the target audio clip and second snore information after the target audio clip based on the initial audio clips;
determining a confidence value for the target audio clip based on the first snore information and the second snore information, wherein the confidence value is configured to represent a possibility that the target audio clip is an abnormal sleep audio clip; and
determining whether the target audio clip is the abnormal sleep audio clip based on the confidence value of the target audio clip.

2. The method of claim 1, wherein determining the target audio clip matching the preset sleep state from the initial audio clips comprises:
obtaining a sleep event recognition result corresponding to each initial audio clip by inputting the initial audio clips into a preset sleep event recognition model;
for each initial audio clip, determining a sleep state recognition result of the initial audio clip in response to the sleep event recognition result corresponding to the initial audio clip matching a preset sleep event, wherein the sleep state recognition result comprise the preset sleep state; and
determining the initial audio clip as the target audio clip in response to a sleep state of the initial audio clip being the preset sleep state.

3. The method of claim 2, wherein determining the sleep state recognition result of the initial audio clip, comprises:
extracting an audio feature of the initial audio clip; and
determining the sleep state identification result of the initial audio clip based on the audio feature of the initial audio clip.

4. The method of claim 2, wherein,
the preset sleep event comprises a snoring event and a breathing event; and
the preset sleep state comprises hypopnea and apnea.

5. The method of claim 1, wherein determining the first snore information before the target audio clip and the second snore information after the target audio clip based on the initial audio clips comprises:
obtaining, in the initial audio clips, a first audio clip before the target audio clip and a second audio clip after the target audio clip; and
extracting, in the first audio clip, the first snore information before the target audio clip, and extracting, in the second audio clip, the second snore information after the target audio clip.

6. The method of claim 1, wherein determining the confidence value for the target audio clip based on the first snore information and the second snore information comprises:
determining an abnormal snore intensity based on the first snore information and the second snore information; and
determining the confidence value for the target audio clip based on the abnormal snore intensity, the preset sleep state corresponding to the target audio clip and a duration corresponding to the preset sleep state.

7. The method of claim 1, wherein determining whether the target audio clip is the abnormal sleep audio clip based on the confidence value of the target audio clip comprises:
determining that the target audio clip is the abnormal sleep audio clip in response to the confidence value of the target audio clip being greater than a threshold.

8. The method of claim 1, wherein the method further comprises:
obtaining a historical abnormal clip based on a sleep state of the abnormal sleep audio clip, wherein the historical abnormal clip is an abnormal clip determined by a user operation; and
determining whether the abnormal sleep audio clip is a true abnormal clip based on the historical abnormal clip.

9. The method of claim 8, wherein determining whether the abnormal sleep audio clip is the true abnormal clip based on the historical abnormal clip comprises:
obtaining a first audio feature of the historical abnormal clip and obtaining a second audio feature of the abnormal sleep audio clip; and
determining that the abnormal sleep audio clip is the true abnormal clip in response to a similarity between the first audio feature and the second audio feature satisfying a preset condition.

10. The method of claim 1, further comprising:
obtaining and displaying sleep aid device information and/or medical aid resource information corresponding to the abnormal sleep audio clip.

11. The method of claim 1, further comprises:
generating a sleep curve based on the abnormal sleep audio clip and displaying the sleep curve, and labeling the abnormal sleep audio clip in the sleep curve, wherein the sleep curve is used to represent sleep states of a user at different time points.

12. An electronic device, comprising:
at least one processor; and
a memory communicatively coupled to the at least one processor; wherein,
the memory stores instructions executable by the at least one processor, when the instructions are executed by the at least one processor, the at least one processor is caused to implement a method for recognizing an abnormal sleep audio clip, the method comprising:
obtaining a plurality of initial audio clips collected by a sensor, and determining a target audio clip matching a preset sleep state from the initial audio clips, wherein the preset sleep state represent an abnormal sleep state;
determining first snore information before the target audio clip and second snore information after the target audio clip based on the initial audio clips;
determining a confidence value for the target audio clip based on the first snore information and the second snore information, wherein the confidence value is configured to represent a possibility that the target audio clip is an abnormal sleep audio clip; and
determining whether the target audio clip is the abnormal sleep audio clip based on the confidence value of the target audio clip.

13. The electronic device of claim 12, wherein determining the target audio clip matching the preset sleep state from the initial audio clips comprises:
obtaining a sleep event recognition result corresponding to each initial audio clip by inputting the initial audio clips into a preset sleep event recognition model;
for each initial audio clip, determining a sleep state recognition result of the initial audio clip in response to the sleep event recognition result corresponding to the initial audio clip matching a preset sleep event, wherein the sleep state recognition result comprise the preset sleep state; and
determining the initial audio clip as the target audio clip in response to a sleep state of the initial audio clip being the preset sleep state.

14. The electronic device of claim 13, wherein determining the sleep state recognition result of the initial audio clip, comprises:
extracting an audio feature of the initial audio clip; and
determining the sleep state identification result of the initial audio clip based on the audio feature of the initial audio clip.

15. The electronic device of claim 13, wherein,
the preset sleep event comprises a snoring event and a breathing event; and
the preset sleep state comprises hypopnea and apnea.

16. The electronic device of claim 12, wherein determining the first snore information before the target audio clip and the second snore information after the target audio clip based on the initial audio clips comprises:
obtaining, in the initial audio clips, a first audio clip before the target audio clip and a second audio clip after the target audio clip; and
extracting, in the first audio clip, the first snore information before the target audio clip, and extracting, in the second audio clip, the second snore information after the target audio clip.

17. The electronic device of claim 12, wherein determining the confidence value for the target audio clip based on the first snore information and the second snore information comprises:
determining an abnormal snore intensity based on the first snore information and the second snore information; and
determining the confidence value for the target audio clip based on the abnormal snore intensity, the preset sleep state corresponding to the target audio clip and a duration corresponding to the preset sleep state.

18. The electronic device of claim 12, wherein determining whether the target audio clip is the abnormal sleep audio clip based on the confidence value of the target audio clip comprises:
determining that the target audio clip is the abnormal sleep audio clip in response to the confidence value of the target audio clip being greater than a threshold.

19. The electronic device of claim 12, wherein the method further comprises:
obtaining a historical abnormal clip based on a sleep state of the abnormal sleep audio clip, wherein the historical abnormal clip is an abnormal clip determined by a user operation; and
determining whether the abnormal sleep audio clip is a true abnormal clip based on the historical abnormal clip.

20. A non-transitory computer-readable storage medium having computer instructions stored thereon, wherein the computer instructions are configured to cause a computer to implement ta method for recognizing an abnormal sleep audio clip, the method comprising:
obtaining a plurality of initial audio clips collected by a sensor, and determining a target audio clip matching a preset sleep state from the initial audio clips, wherein the preset sleep state represent an abnormal sleep state;
determining first snore information before the target audio clip and second snore information after the target audio clip based on the initial audio clips;
determining a confidence value for the target audio clip based on the first snore information and the second snore information, wherein the confidence value is configured to represent a possibility that the target audio clip is an abnormal sleep audio clip; and
determining whether the target audio clip is the abnormal sleep audio clip based on the confidence value of the target audio clip.
